(19) United States
(12) Patent Application Publication
(10) Pub. No.: US 2008/0171950 A1
(43) Pub. Date: Jul. 17, 2008

(54) Systems and methods for a pregnancy monitoring device

(75) Inventor: Jeff Franco, Clarksville, MD (US)

Correspondence Address: ROPES & GRAY LLP, PATENT DOCKETING 39/41, ONE INTERNATIONAL PLACE, BOSTON, MA, 02110-2624, US

(73) Assignee: Genisent International Inc., Baltimore, MD (US)

(21) Appl. No.: 12/002,667

(22) Filed: Dec. 18, 2007

Related U.S. Application Data
(60) Provisional application No. 60/875,683, filed on Dec. 18, 2006.
(60) Provisional application No. 60/925,057, filed on Apr. 17, 2007.

(51) Int. Cl. A61B 5/053; A61M 5/172

(52) U.S. Cl. 600/547; 604/66

(57) ABSTRACT

Systems and methods are provided for characterizing electrical activity of a patient for making a pregnancy-related diagnosis. The system includes a wearable device for measuring an electrical impedance of a cervical tissue of the patient based on a signal applied to the cervical surface by the device. The system also includes a transmitter coupled to the wearable device for transmitting the measured electrical impedance of the cervical tissue to an analysis system for making a pregnancy-related diagnosis based on the impedance data.

### REFERENCE TO RELATED APPLICATIONS

This Application claims the benefit of U.S. Provisional Application Ser. No. 60/875,683 filed on Dec. 18, 2006 and U.S. Provisional Application Ser. No. 60/925,057 filed on Apr. 17, 2007, the entire contents of which are incorporated herein by reference.

### BACKGROUND OF THE INVENTION

Many women have difficulty carrying a baby to full term. They suffer from miscarriages and have an increased risk of premature labor. In many cases, miscarriages and premature labor can be prevented if preventative measures are taken. However, a medical professional needs to know that the miscarriage or premature labor is likely to happen in order to intervene.

Remote or in-home monitoring of physiological conditions associated with a pregnant woman has become more frequent and prevalent in recent years. However, many labor monitoring systems are inaccurate, often providing predictions that result in undesirable outcomes such as slow and difficult birth or premature delivery. In addition, these systems tend to introduce discomfort to the woman by limiting her mobility while she is being monitored.

The scheduling of births at full term is becoming more prevalent, for example, through planned induction of labor. However, inducing labor too early may lead to extended labor times and additional risks of complications.

### SUMMARY OF THE INVENTION

The system and apparatus described herein provide a less invasive, and less intrusive means for a medical professional to monitor pregnant women with higher risk of miscarriage or premature labor to detect the warning signs of miscarriage or premature labor early enough to intervene. The systems and methods described herein also provide a less invasive and intrusive means for monitoring the later stages of pregnancy to provide a better prediction as to when labor inducement will be effective with reduced risk of complications.

The systems and methods described herein are related to a wearable device for measuring electrical impedance of a pregnant patient's cervical tissue. The system includes a wearable device and a transceiver. The transceiver is coupled to the wearable device for transmitting the measured electrical impedance of the cervical tissue to an analysis system for making a pregnancy-related diagnosis based on the impedance data. The transceiver wirelessly transmits the measured electrical impedance to the analysis system. In some embodiments, the analysis system is local to the patient. In other embodiments, the analysis system is geographically separated from the patient.

The wearable device is preferably made from a waterproof, substantially flexible, and non-irritating material. The device has electrodes disposed on at least one of its surfaces, where some of the electrodes are adapted to send an electrical signal to the patient's cervical tissue and other electrodes are used to sense electrical impedance based on the transmitted electrical signal. The electrical impedance is inversely correlated to a desirability of labor induction, a shorter labor time period, a possibility of requiring a C-section, and labor-related complications. In one implementation, the wearable device is a ring-shaped structure having a diameter between about 50 mm to about 55 mm and a thickness of about 4 mm. In another implementation, the wearable device is a cap structure having two open ends and is adapted to fit over a cervical opening of the patient without sealing the cervical opening. In yet another implementation, the wearable device is a clip structure that may be stapled to the cervical tissue and is adapted to include a side having the electrodes disposed thereon that contact the cervical tissue. In another implementation, the wearable device is a strip structure having a plurality of electrodes in communication with one another. The strip structure has a flexible portion that allows the device to contour to a patient's cervix. The strip structure also has a plurality of arrays of micro-needle sized and shaped to secure the strip to the cervical surface of the patient. In some embodiments, the strip is adapted to the patient by stretching of the flexible portion during application of the strip to a vaginal surface, and upon release, transferring the tension released on the flexible portion to the micro-needle, thereby adhering the strip to the vaginal surface. In general, the wearable device comfortably inter-fits within a vaginal region, preferably adjacent to, embedded in, or surrounding the cervix, of the patient for a period of time. This time period may be three weeks, three months, six months, or nine months. In some embodiments, the time period exceeds one month.

In certain embodiments, the wearable device includes a power unit, a memory unit for storing the measured impedance. In certain embodiments, the system may further include a signal processing unit wirelessly coupled to the wearable device for enhancing signal quality of the electrical impedance data and for forwarding the enhanced electrical impedance to the analysis system. In some embodiments, the transceiver sends the measured electrical impedance in real-time or the measured electrical impedance data stored in the memory unit to the analysis system.

In one exemplary application area, the analysis system is used to make pregnancy-related diagnoses including, for instance, labor prediction, prediction of a pregnancy-related complication, such as pre-term labor, and prediction of a suitable delivery approach. Suitable delivery approaches typically include non-induced vaginal birth, cesarean section, and labor induction. In some implementations, these pregnancy-related diagnoses are made by comparing the measured impedance data to data in a database. Data in the database may include historical cervical tissue measurements of the patient or cervical tissue measurements of a group of women having similar physiological profiles as the patient. In addition, the analysis system is capable of determining at least one of time, duration, and dosage amount of an agent to deliver to the patient via the wearable device based on an evaluation of the collected impedance data.

In certain embodiments, the analysis system compares each received impedance value to a predetermined impedance threshold for performing a Cesarean-section. If the impedance value is greater than the Cesarean-section threshold, the analysis system diagnosis the option of having a Cesarean-section at a later date. If the impedance value is lower than the Cesarean-section threshold, the received impedance value is compared to a threshold for inducing labor in the patient. In cases where the impedance value is greater than the threshold for inducing labor, the analysis system diagnosis to induce labor in a patient at a later date. In cases where the impedance value is less than or equal to the induction threshold, the analysis system diagnosis an immediate labor inducement.

In certain embodiments, the system includes at least one reservoir containing a drug for treating a pregnancy-related complication. The reservoir may be affixed onto the surface of the device having electrodes for measuring an electrical impedance of a cervical tissue of the patient. The system also includes an analysis system that is coupled to the wearable device. In addition, the analysis system controls a release of the drug from the reservoir based on the electrical impedance data.

In certain examples, the analysis system, whether remote or local, incorporates numerous service functions for performing at least one of delivering medical care to the patient, alerting personnel pertinent to the patient, and making a medical suggestion to the patient regarding her overall health based on an evaluation of the impedance data.

### BRIEF DESCRIPTION OF THE DRAWINGS

These and other features and advantages will be more fully understood by the following illustrative description with reference to the appended drawings in which the drawings may not be drawn to scale.

FIGS. 1a-b illustrate exemplary ring-shaped devices for measuring electrical impedance of cervical tissues, according to an illustrative embodiment of the invention.

FIGS. 2a-b illustrate exemplary cap devices for measuring electrical impedance of cervical tissues, according to an illustrative embodiment of the invention.

FIG. 3 illustrates an exemplary strip-shaped device for measuring electrical impedance of cervical tissues, according to an illustrative embodiment of the invention.

FIGS. 4a-c illustrates an exemplary clip device for measuring electrical impedance of cervical tissues, according to an illustrative embodiment of the invention.

FIG. 5 illustrates the ring-shaped device shown in FIG. 1b having a reservoir, according to an illustrative embodiment of the invention.

FIGS. 6a-b illustrate a top and a side view, respectively, of a strip-shaped device for measuring electrical impedance having a reservoir, according to an illustrative embodiment of the invention.

FIG. 7 illustrates a schematic diagram of a pregnancy monitoring system having local and remote monitoring capabilities, according to an illustrative embodiment of the invention.

FIG. 8 illustrates an exemplary decision-making process for determining a suitable delivery approach for a pregnant patient based on measured cervical impedance values taken from the patient.

### DESCRIPTION OF CERTAIN ILLUSTRATIVE EMBODIMENTS

The present invention provides methods and systems for a conveniently wearable cervical monitoring device that is capable of periodically or continuously taking measurements of cervical impedance in pregnant women and transmitting the resulting measurements to a local and/or a remote location for analysis and monitoring of pregnancy and/or labor conditions. The following detailed description of the invention refers to the accompanying drawings. The following description does not limit the invention, and the various examples set out below and depicted in the figures are merely provided for the purposes of illustrating certain examples of these systems and methods and for describing examples of such systems and methods.

There exists a noticeable difference in the electrical impedance of cervical tissues of pregnant women in various stages of their pregnancies. A healthcare professional may use this information to monitor a patient's progression of pregnancy, to predict, at different stages of the patient's pregnancy, a date of delivery and the likelihood of pre-term labor. In addition, the healthcare professional may use the predictions to schedule a suitable delivery procedure with the goal of minimizing the length of labor and birth-related complications. The healthcare professional may also use the measured tissue impedance information to objectively determine, in real-time, an appropriate delivery procedure among a variety of delivery options available to the patient. These delivery procedures include, for example, cesarean section (C-section), labor induction, non-induced vaginal labor. The impedance-measuring device is portable and conveniently wearable by the patient so that the patient's condition can be periodically or continuously monitored without causing her much discomfort or impeding her routine activities. Furthermore, the device preferably is also able to wirelessly transmit the detected impedance data to a local or a remote system for analysis. Based on this analysis, the patient or a healthcare professional is alerted of detected complications that may arise during the course of the pregnancy. In certain embodiments, the device is used to perform on-demand vaginal delivery of drugs, such as progesterone for delaying the onset of labor. The time, duration and dosage amount of drugs delivered to the woman can be regulated by the local and/or remote monitoring systems of the present invention.

In general, animal tissue produces electrical current patterns after being stimulated by a low-voltage current source. These patterns are measurable over a range of frequencies for determining intracellular and extra-cellular properties associated with the tissue. With respect to cervical tissue in pregnant women, a higher cervical impedance correlates to a longer period to the onset of labor. This observation is indicative of the fact that resistivity of a pregnant cervix decreases as the cervix undergoes a ripening process which changes its hydration and collagen content. More specifically, changes in resistivity measurements reflect changes in intracellular and extra-cellular fluid in the cervix as well as changes in cervical cell orientations. Hence, impedance measurements taken over various stages of a woman's pregnancy may be used to provide near-term or real-time detection of likely pre-mature labor and to predict a date of delivery. In addition, the measured electrical impedance can be used to determine an appropriate delivery approach such as vaginal delivery without induction, C-section or labor induction. Typically, lower electrical impedance tends to suggest a favorable response to labor induction, a shorter labor time period, a lower possibility of requiring a C-section, and fewer labor-related complications.

Hence, the bio-impedance measuring device in combination with the local/remote monitoring system of the present invention is useful to pregnant women in general, but particularly useful to women who have a history of miscarriage or pre-term birthing. These women can be closely monitored using this device during, for example, the last three weeks, the last three months, the last six months, or the entire length of their pregnancies to attempt to identify signs of pre-term labor early enough to intervene.

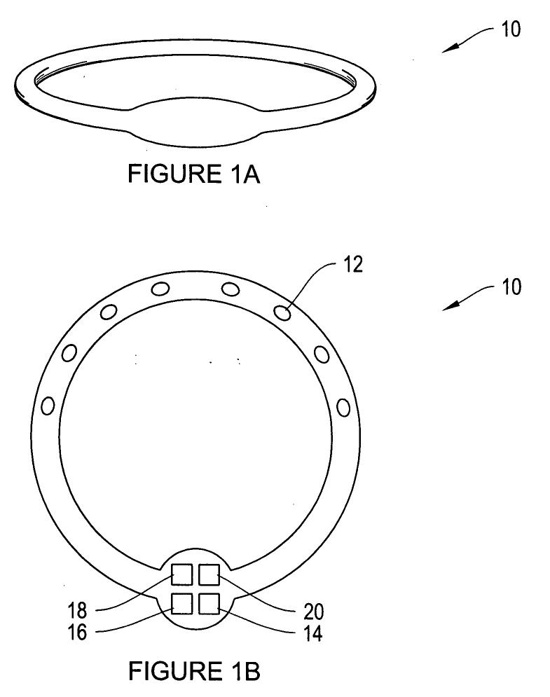

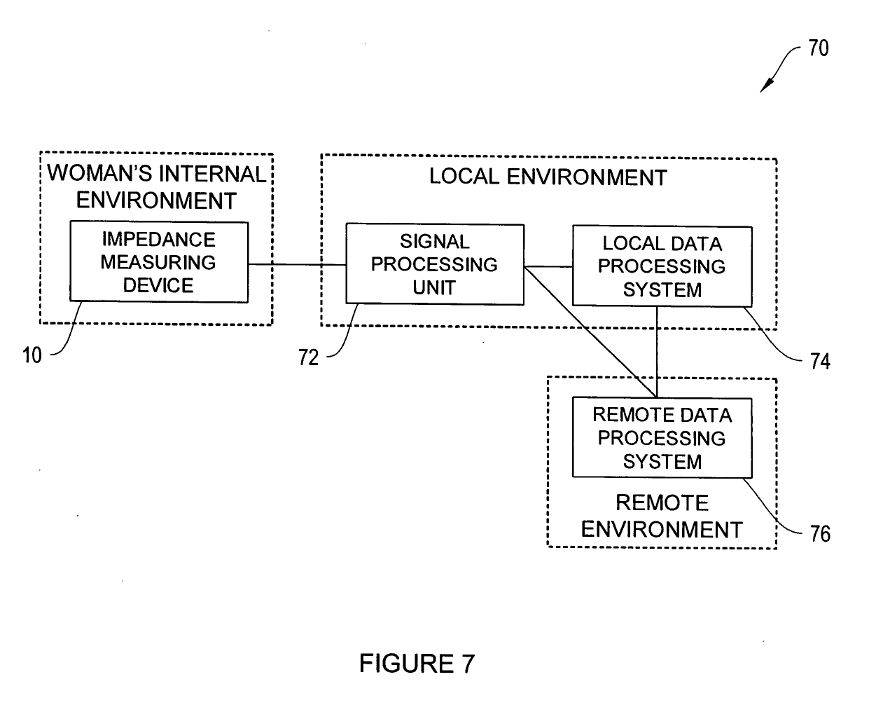

FIG. 1a depicts an exemplary device 10 suitable for insertion into a woman's vagina to measure electrical impedance of her cervical tissues. The exemplary device is a resilient and flexible ring-shaped structure that is about 50 mm to about 55 mm in diameter and about 4 mm in thickness. The ring may be constructed from an inert and non-irritating material, such as ethylene vinyl acetate (EVA), so that it is safe to remain in a woman's vagina during a long-term monitoring process (e.g., 90-180 days) and does not cause infection. Two or more electrodes are disposed on the surface of the ring and are situated such that they are in contact with the woman's cervical tissue. The electrodes may be arranged such that a portion of the electrodes form an electrical circuit when in contact with the cervical tissue to deliver signals or current to the tissues. The remaining electrodes can sense a voltage or other electrical characteristic of the tissue when the signals or current is flowing through the tissue. For instance, four electrodes may be disposed on the surface of the ring in a tetra-polar configuration, where two electrodes are used to deliver electrical signals and the other two are used to sense the delivered signals. A wireless transceiver may also be coupled to the ring-shaped surface for sending a measurement of the voltage or electrical characteristic from the electrodes to a data-receiving system external to the woman's body. The data receiving system will be described below in relation to FIG. 7. Alternative embodiments may have fewer or more electrodes or more transceivers in various arrangements on the device surface.

FIG. 1b shows another view of ring-shaped bio-impedance measuring device 10. In addition to the numerous electrodes disposed on the surface of the ring, the ring also includes a power unit 20, a memory unit 16, a transceiver 14, and a processor 18 for coordinating the operation of the device. The power unit 20 is preferably a battery. The memory unit 16 is used to store measured impedance data. The transceiver 14 is used to transmit measured impedance readings, either in real-time or with data stored in the memory unit to an external location for storage and analysis. The processor 18 is preferably a special purpose processor, such as an application specific integrated circuit in electrical communication with the transceiver 14, the electrodes 12, and the memory 16.

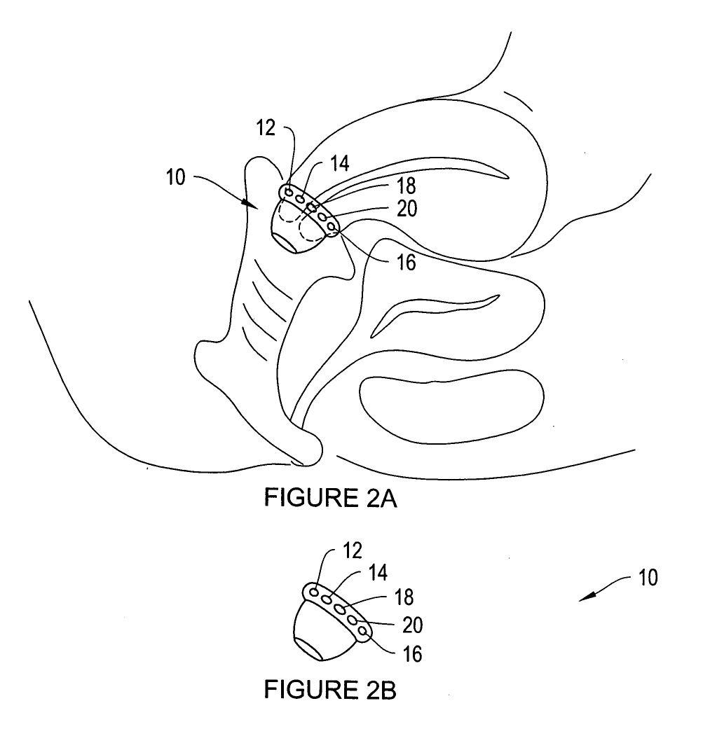

FIG. 2a depicts another exemplary wearable device 10 for measuring electrical impedance of a woman's cervical tissues. As shown, the device 10 is constructed as a thimble-shaped cap that is insertable into a woman's vagina and fits snugly over her cervix such that minimal mucus is accumulated between the cap and her cervix. The cap may be made of latex, silicone, or other non-irritating and pliable material. In an alternative configuration, as illustrated in FIG. 2b, the cervical cap has two open ends so that the cervical opening is not sealed by the cap. The cervical caps, like the ring-shaped device, include a processor 18, a memory 16, a electrode 12, a transceiver 14, and a power unit 20 for measuring and outputting cervical impedance data. The electrodes 12 are preferably coupled to an interior surface of the cap that substantially contacts the cervical tissue.

The impedance-measuring devices, as described above with respect to FIGS. 1a-b, and 2a-b may easily be inserted into a woman's body without the assistance of a healthcare professional. Thus, the devices may serve as a part of a convenient pregnancy monitoring system to in-home patients or outpatients. In operation, the pregnant woman inserts a device, such as the ones described above, into her vagina where the device conforms to fit comfortably and remains in place until it is removed by the woman. These devices are also adapted to be waterproof.

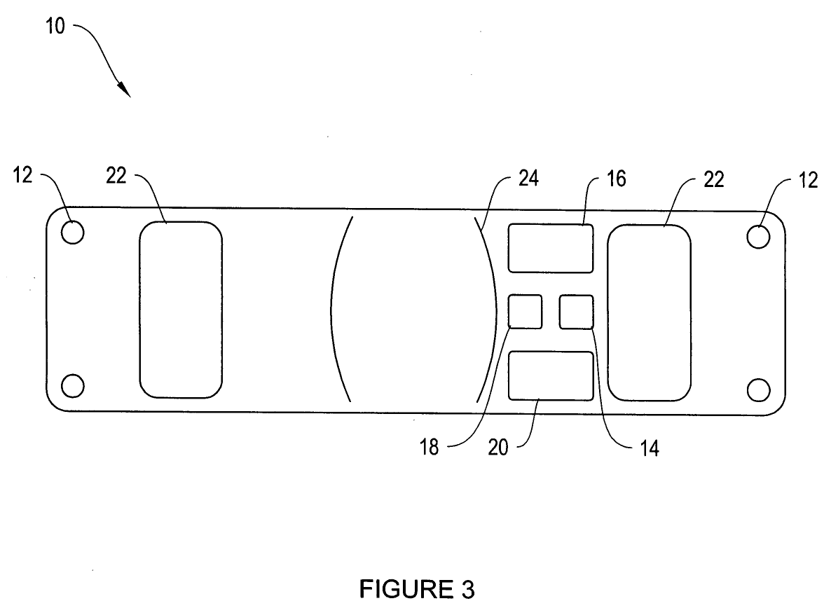

FIG. 3 depicts yet another exemplary tissue bio-impedance measuring device 10. The device 10 is constructed as a strip having a flexible portion 24 that allows the device to contour to a patient's cervix. The device includes two arrays of micro-needles 22 that are adapted to secure the device to a cervical surface of the patient. The microneedles 22 are preferably barbed to increase their adhesive effect and are short enough to not reach the nerve endings of the cervical tissue to avoid generating a pain response. Two ends of the strip are provided with electrodes 12 for sending current into the cervical tissue and for measuring the cervical impedance. Another portion of the strip may be used to store, for example, a power unit 20, a memory unit 16, a transceiver 14, and a processor 18 for coordinating the operation of the device. The flexible portion is also preferably elastic such that the device can be applied by stretching the strip slightly, applying it to the cervix, and releasing it. The tension provided by the flexible portion 24 of the strip is transferred to the microneedles 22 embedded in the tissue, increasing their adhesive properties. In another implementation, the microneedles 22 are replaced with small hooks. As the cervix has few nerve endings and is not particularly sensitive to pain, the hooks need not be on the scale of microneedles to maintain a relatively painless adhesion.

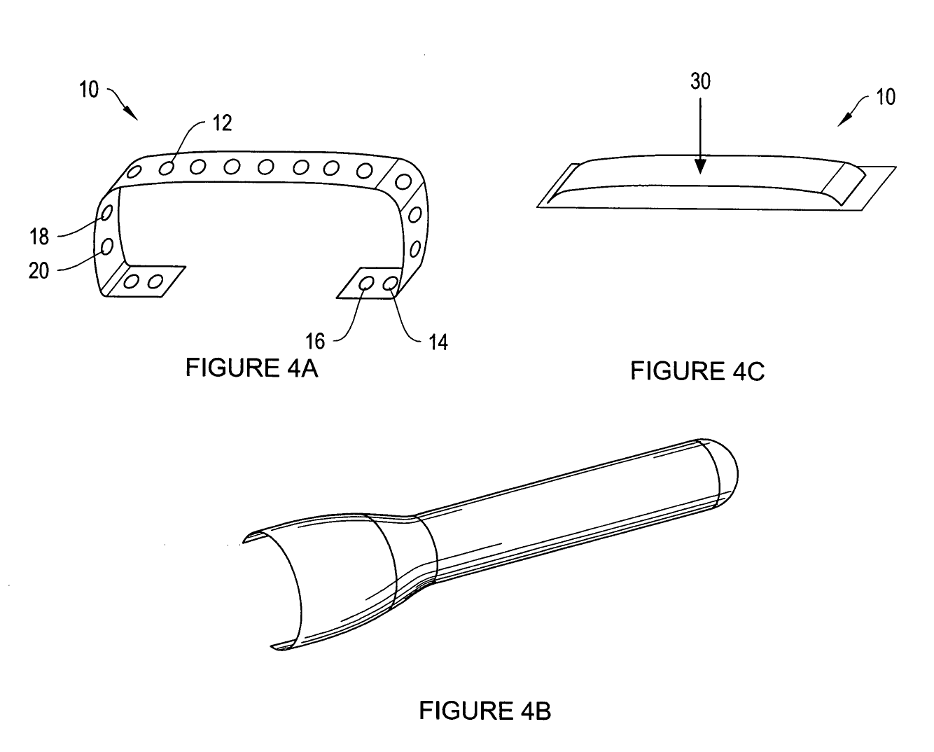

FIG. 4a shows yet another wearable bio-impedance monitoring device 10. This device is configured as a surgical staple, such as a hemaclip, that can be implanted into the patient's cervical tissue. Electrodes 12, a power source 20, a processor 18, a memory 16, and a transceiver 14 are coupled a surface of the staple to measure and transmit tissue impedances. The electrodes are preferably located on the surface of the staple that is in contact with the cervical tissue wherefrom the measurements are taken. This clip-like device is implantable into a woman's cervical tissue by a physician using, for instance, a surgical stapler, as shown in FIG. 4b. It is also easily removable by the physician using a surgical remover that operates by applying pressure to the center 30 of the staple while simultaneously lifting from the staple's edges as depicted in FIG. 4c.

In one implementation, labor or pre-term labor predictions are made based on changes in the patient's cervical impedance or a rate of change of the patient's cervical impedance. In certain examples, these evaluations are made by comparing the patient's impedance measurements to a database of impedance values. The database of impedance values may be compiled based on the patient's historical cervical measurements taken from her past pregnancies or from statistical impedance data taken from women who have similar physiological profiles as the patient. If pre-term labor is predicted, a physician is able to prescribe an appropriate dose of progesterone to the patient to delay the onset of her labor. Other drugs that are effective in alleviating conditions associated with preterm labor include, for example, beta-agonists (e.g. terbutaline, ritodrine and isoxuprine), magnesium sulfate, nifedipine (e.g. procardia), and indomethacin (e.g. indocin). However, any drugs can be delivered to the patient using the wearable devices of the present invention. The term “drug” may refer to an agent that possess therapeutic, prophylactic, or diagnostic properties in vivo when administered to patients. In general, the amount of drug can be selected by one of skill in the art, based, for example, on the particular drug, the desired effect of the drug at the planned release level, and the time span over which the drug is released. In certain examples, the type and amount of drugs administered to the patient are also dependent on her medical history which may reveal, for example, certain drugs that the patient is allergic to.

In some embodiments, progesterone is deliverable to the patient by injection, intra-vaginally or orally, and the dosage level is determined by a combination of the patient's gestational age and her impedance value, which correlates to a state of ripening of the patient's cervix.

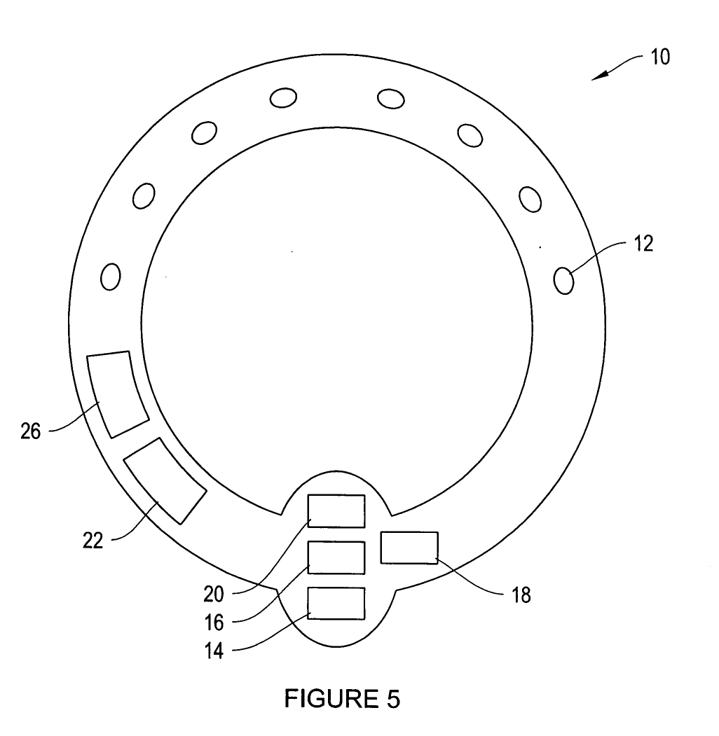

In another example, drugs can be eluted on-demand from a bio-impedance measuring device 10, such as the devices depicted in FIGS. 1a-b, 2a-b, 3, and 4a. Drug elution is controllable by the remote monitoring system based on thorough analysis of the patient's impedance readings. FIG. 5 shows an embodiment of a device having a reservoir 26 containing a drug for treating pregnancy-related complications. In one embodiment, the reservoir 26 can be integrally formed into the ring-shaped structure. In an alternative embodiment, the reservoir 26 is affixed onto a surface of the ring-shaped structure. The reservoir 26 may comprise a volume surrounded by one or more walls, such as a balloon-like pouch or comprise a porous material, such as a sponge, which is able to retain, for example, a drug in liquid form until the material is compressed. In certain embodiments, the reservoir additionally includes an outlet that has an open or closed state for controllably dispensing the drug contained therein. In general, the volume of the reservoir is configured to provide sufficient medication to a patient for a day, three weeks, three months, six months, or any pre-determined length of time during the patient's pregnancy.

The reservoir 26 may be made from similar material as the ring and is preferably formed from a deformable or elastic material. However, in certain embodiments, the reservoir 26 may be substantially rigid. The reservoir 26 may be formed from one or more polymers, metals, ceramics, or combinations thereof. In addition, the reservoir 26 may be constructed to keep the drug composition free of contaminants and degradation-enhancing agents. For example, the reservoir 26 is able to exclude light when the drug composition contains photo-sensitive materials and may include an oxygen barrier material to minimize exposure of drugs sensitive to oxidation. Also, the reservoir 26 is able to keep volatile materials from entering therein to prevent any alteration of the composition of the drug that may render it undeliverable to the patient.

FIG. 5 also shows a device including, in addition to a reservoir 26, a power unit 20, a memory 16, a processor 18, and a transceiver 14 for coordinating the operation of the device. The power unit 20 is preferably a battery. The processor 18 is preferably a special purpose processor, such as an application specific integrated circuit in electrical communication with the transceiver 14, the electrodes 12, the reservoirs 26, and the memory 16. The transceiver 14 is configured to receive a signal from the local/remote monitoring system, where such signal regulates the time and amount of drugs dispensable to the patient from the reservoir. The memory 16 is used to store impedance data measured by the electrodes as well as drug delivery instructions received from the local or remote monitoring system. The signals stored in the memory 16 may be used by the processor 18 to coordinate the dispensing of drugs from specific reservoirs 26 at designated times and durations. This is accomplished, for example, by controlling the movement of a plunger or gating mechanism coupled to each reservoir for releasing the drug stored therein. In some embodiments, the function of the transceiver may be separated to a stand-alone transmitter and a receiver. In some embodiments, if the bio-impedance measuring device does not need to receive data, the device may include a stand-alone transmitter instead of a transceiver.

In some embodiments, the device of FIG. 5 may additionally include at least one micro-needle 22 having one end coupled to the reservoir 26 and another end configured to penetrate into biological tissue with minimal or no damage, pain, or irritation to the tissue. The micro-needle 22 may thus be used to deliver drugs from the reservoir to a woman's cervical tissue at clinically relevant rates. The micro-needles 22 are preferably hollow and barbed to increase their adhesive effect and are short enough to not reach the nerve endings of the cervical tissue, to avoid generating a pain response. The micro-needles 22 can be oriented perpendicularly or at any angle with respect to a surface of the ring structure to which the micro-needles are attached. In an alternative implementation, the micro-needles 22 are replaced with small hollow hooks. As the cervix has few nerve endings and is not particularly sensitive to pain, the hooks need not be on the scale of micro-needles to maintain a relatively painless adhesion while introducing drugs into the cervical tissue of a pregnant patient. In some embodiments, the reservoir 22 may be integrated or affixed to the surfaces of the bio-impedance measuring devices depicted in FIGS. 2a, 3, and 4a.

According to one exemplary drug delivery method, a reproducible pressure is applied to the reservoir to expel its content at a site of administration via the one or more needles to which the reservoir is coupled. A similar drug delivery methodology is disclosed in U.S. Pat. No. 6,611,707, which is incorporated herein by reference in its entirety. The reproducible pressure may be controllably supplied by a plunger that is adapted to compress the reservoir upon the device receiving a trigger or release signal from the local/remote system of the present invention. The amount of drug expelled from the reservoir is thus dependent on the amount of force applied to the reservoir as well as the length of time to which the force is applied. In addition, drugs may be released from the reservoir at clinically relevant rates proportional to the rate with which the force is applied to the reservoir. In another exemplary drug delivery approach, the outlet of the reservoir can be controllably regulated to assume either an open, closed, or partially open state. Particularly, in the closed state, the outlet is adapted to confine the drug in its reservoir such that the drug does not leak out and contact the cervical tissue of the patient. In the open state, the outlet permits the drug to flow from the reservoir, through the micro-needles, and into a target cervical tissue site for the precise administration of labor-related treatments. Moreover, the outlet may provide a specific drug flow rate by setting, for example, the degree to which the outlet is opened. Hence the amount of drug dispensed via the outlet may be regulated based on a combination of the drug flow rate and the length of time the outlet is in the open state. Furthermore, the amount of drugs flowing through the micro-needles into the patient's tissue can be set by selecting the effective hydrodynamic conductivity of the micro-needles by, for example, increasing or decreasing the number or diameter of the micro-needles. In other implementations, delivery can be initiated by opening a mechanical gate or valve interposed between the reservoir outlet and the micro-needle inlets. In some embodiments, drugs in the reservoir are released by electrostatic or capillary forces.

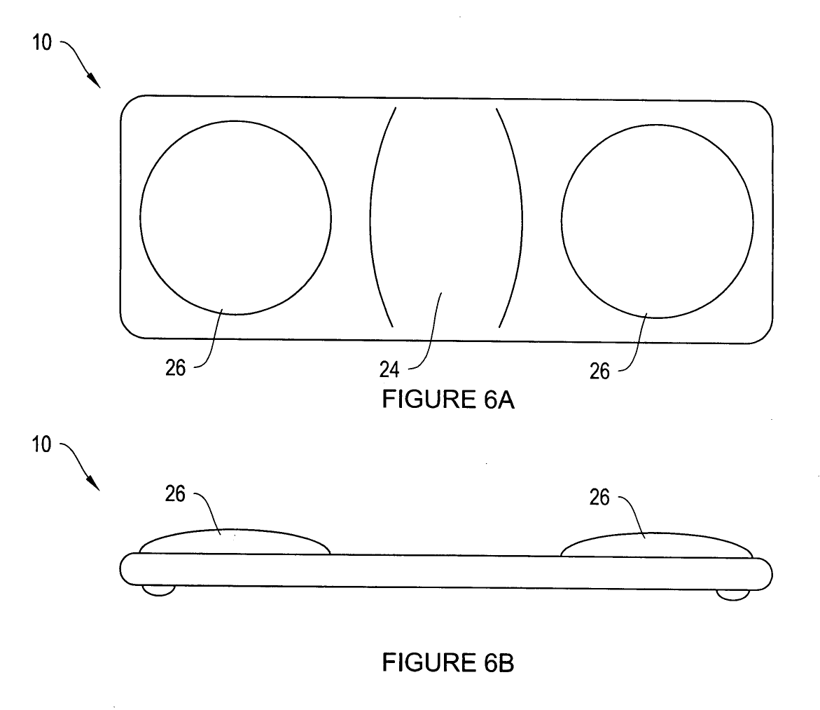

In certain configurations, an impedance-measuring device may include multiple reservoirs 26 for storing different types of drugs or drugs of different concentrations that are likely to be administered to the patient. FIGS. 6a-b show a device 10 having two reservoirs 26 and a flexible portion 24 located between the reservoirs 26. These reservoirs are isolated from one another from a portion of a micro-needle array included in the device. Thus, the device can, for example, be provided to deliver different drugs through different needles, or to deliver the same or different drugs at different rates or at different times, as signaled by the local/remote system based on monitored pregnancy conditions of the patient. Alternatively, the contents of the different reservoirs 26 can be combined with one other so as to allow the materials to mix before being delivered to the patient.

FIG. 7 illustrates a schematic diagram 70 of a pregnancy monitoring system having local and remote monitoring capabilities. The pregnancy monitoring system includes a signal processing unit 72, a local data processing system 74, and a remote data processing system 76. The signal processing unit 72 is wirelessly coupled to the impedance-measuring device 10 to process signals transmitted from the device. This signal processing unit includes, for example, filters, amplifiers, and noise reduction circuitry for clarifying and enhancing the measured signals from the impedance-measuring device. The signal processing unit can also include an internal memory module for storing the processed data and a transceiver for enabling data communication to a local system accessible by the patient, such as an in-home system, or a geographically remote central system for analysis and monitoring of the patient's physiological conditions during her pregnancy. Such signal processing unit may be compact in construction and may be wearable, for example, on a belt, by the patient so as to facilitate her movement and daily activities. The signal processing unit 72 may also be a stand-alone device or integrated into a personal computer or a mobile device, such as a PDA.

In other system configurations, one or more wireless transceivers may be placed at various locations frequented by the patient, for example, at her home or work place, to transmit the raw electrical impedance data from the device to an external location for signal/data processing. In yet other system configurations, a stand-alone communication device is provided that includes, for example, a modem, a transceiver, and an internal memory. This communication device may be plugged into a telephone jack or connected to a computer, for example, via a USB port, to transmit the patient's impedance data to an external location for data processing.

Transmission of the patient's data can be automatically initiated at regular intervals according to pre-programmed instructions in software, hardware, or firmware of the local and remote systems, or manually initiated by the patient, if desired, and can be done in real-time or with data stored in an internal memory unit. Data transmitted can be raw data from the impedance-measuring device or processed data from the signal processing unit.

In certain system configurations, electrical impedance data from at the impedance-measuring device and/or the signal processing unit is transferable to a remote data processing system 76 where predictions on labor or pre-term labor are made using a combination of computerized statistical analysis and expert input. Remote systems generally refer to systems that reside in locations geographically remote and separated from the patient. Hence, remote-monitoring systems offer convenience to those patients who have difficulties getting to a medical center or need extra care due to prior history of labor complications such as pre-term labor.

In one implementation, if analysis of the impedance data indicates the likelihood of pre-term labor, the pregnant woman can be alerted through the system. For example, in embodiments in which the signal processing device is worn on a woman's belt, the device can include a pager component for receiving alerts. Alternatively, the system can contact the pregnant woman via phone with a prerecorded message alerting her to contact her doctor.

In another implementation, raw or processed electrical impedance data is transferable to a local data processing system 74 to provide pregnancy or labor condition evaluation that is readily viewable by the patient or any pertinent personnel for on-site monitoring. These pertinent personnel include, for example, a doctor, nurse, spouse, family member or friend of the patient. The local data processing system 74 may be coupled to other monitoring devices, such as a heart rate monitor, to provide general assessment of the patient's well-being as well as the well-being of the fetus. This information can be passed to the remote monitoring system along with the impedance data.

Hence, the local data processing system 74 and remote data processing system 76 are able to suggest to the patient, based on analysis of collected impedance data and other physiological measurements, certain favorable activities for the patient to perform to improve her overall health. The systems may also alert the patient to see a healthcare professional if an unfavorable trend is detected in the collected data. The local data processing system 74 may reside in a personal computer, a handheld device, a cellular phone or any other communicative devices easily accessible by the patient or the pertinent personnel.

A doctor's diagnosis or advice may be communicated to the patient or pertinent personnel using any communicative means including from the geographically remote system to the local, or in-home, system of the patient. The local data processing system 74 and the remote data processing system 76 may be integrated with other service-related components to perform at least one of automatic ambulance dispatching, automatic calling of the pregnant woman or other pertinent personnel in the event of a detected emergency, and sounding off an alert in the patient's home when abnormalities are detected that need immediate medical attention. In systems in which the process unit is on a belt, an alert can be communicated to the device. These function are provided, for example, through the local and/or remote systems' integration with call centers and hospitals local to the patient.

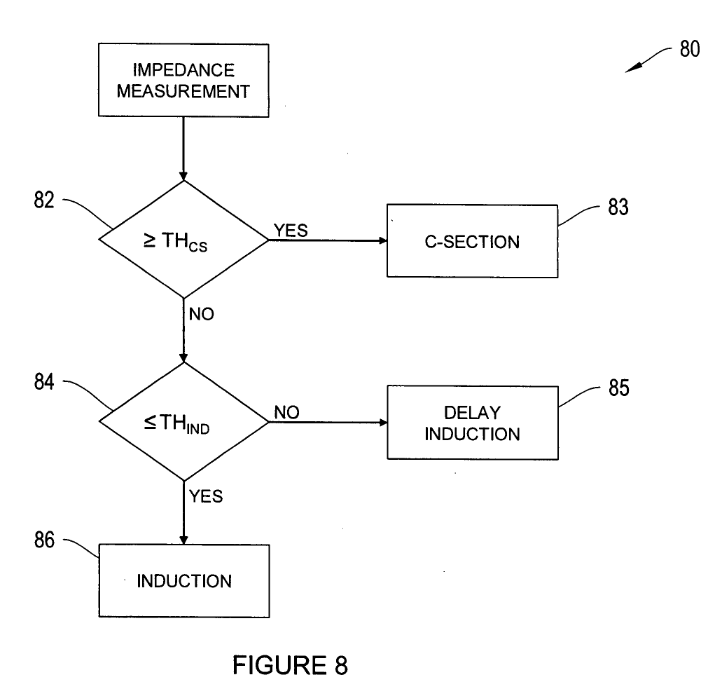

In another implementation, an appropriate delivery approach can be scheduled on the predicted date of delivery. A suitable delivery approach may be, for example, a non-induced vaginal delivery, a drug-induced labor, or a C-section. An illustrative decision-making process 80 as shown in FIG. 8 may be employed on dates prior to the expected delivery date for evaluating future delivery approaches. As depicted, each impedance value received by the system is first compared to a predetermined impedance threshold for performing a C-section, herein referred to as a “C-section threshold”, at step 82 in FIG. 8. If the impedance value is greater than the C-section threshold, the patient is presented with the option of having a C-section at a future date at step 83. In such cases, the relatively high impedance value indicates that a vaginal labor is likely to be particularly long and/or difficult. If the impedance value is lower than the C-section threshold, the impedance value is compared to a threshold for inducing labor in the patient, herein referred to as an “induction threshold”, at step 84 in FIG. 8. If the impedance value is greater than the induction threshold, the physician may decide to induce labor at a future time at step 85. In this case, the patient can remain at home, but with extra monitoring, or be hospitalized for observation. However, if the impedance value is less than or equal to the induction threshold, the physician can decide to immediately induce labor in the patient at step 86.

The C-section threshold 82 and the induction threshold 84 may be individual impedance measurements or statistical means or averages of impedance measurements determined from a large sample of women. These threshold values are adapted to change depending on the date prior to the expected delivery date. In certain implementations, one set of thresholds may be utilized to evaluate labor-related characteristics in every patient. In certain implementations, thresholds are adjusted to correlate to labor characteristics in individual patients or patients having similar physiological profiles based on, for example, their age, health, or race. In certain implementations, the thresholds used for labor evaluations and predictions are adjusted based on the inducement technique desired by the patient. For example, the induction threshold for chemical inducement using pitocin may be different than the threshold for mechanical inducement using forceps.

The invention provides methods and systems for continuously monitoring and predicting, from a local or a remote location and at different stages of a patient's pregnancy, the occurrences of labor or pre-term labor based on electrical impedance measurements of the patient's cervical tissues taken from a wearable impedance measuring device. One skilled in the art will appreciate that the invention can be practiced by other than the described embodiments, which are presented for purposes of illustration and not of limitation.

What is claimed is:

1. A system for characterizing electrical activity of a patient, comprising:
a wearable device for measuring an electrical impedance of a cervical tissue of the patient based on a signal applied to the cervical surface by the device; and
a transmitter coupled to the wearable device for transmitting the measured electrical impedance of the cervical tissue to an analysis system for making a pregnancy-related diagnosis based on the impedance data.

2. The system of claim 1, wherein the transmitter wirelessly transmits the measured electrical impedance to the analysis system.

3. The system of claim 1, wherein the electrical impedance is inversely correlated to a desirability of labor induction, a shorter labor time period, a possibility of requiring a C-section, and labor-related complications.

4. The system of claim 3, wherein a lower electrical impedance indicates labor induction, a shorter labor time period, a lower likelihood of requiring a C-section, and fewer labor-related complications.

5. The system of claim 3, wherein a higher electrical impedance indicates a less favorable labor induction outcome, a longer labor time period, a greater likelihood of requiring a C-section, and a greater likelihood of labor-related complications.

6. The system of claim 1, wherein the wearable device includes a power unit, a memory unit for storing the measured impedance, and a processor.

7. The system of claim 6, wherein the transmitter sends the measured electrical impedance in real-time or the measured electrical impedance stored in the memory unit to the analysis system.

8. The system of claim 1, wherein the continuously wearable device is one of a ring structure, a cap structure, a clip structure, and a strip structure having a plurality of electrodes disposed thereon and adapted to comfortably inter-fit within a vaginal region of the patient for a time period.

9. The system of claim 8, wherein the time period exceeds one month.

10. The system of claim 8, wherein the continuously wearable device is made from a waterproof, substantially flexible, and non-irritating material.

11. The system of claim 8, wherein the wearable device comprises a ring structure, which has a diameter between about 50 mm to about 55 mm and a thickness of about 4 mm.

12. The system of claim 8, wherein the wearable device comprises a cap structure, which has two open ends and is adapted to fit over a cervical opening of the patient without sealing the cervical opening.

13. The system of claim 8, wherein the wearable device comprises a clip structure, which is stapled to the cervical surface and includes a side having the plurality of electrodes disposed thereon while substantially contacting the cervical surface.

14. The system of claim 8, wherein the wearable device comprises a strip structure, which includes:
a plurality of electrodes in communication with one another;
a flexible portion that allows the device to contour to a patient's cervix; and
a plurality of arrays of micro-needle sized and shaped to secure the strip to the cervical surface of the patient.

15. The system of 14, wherein the strip is adapted for application to the patient by stretching of the flexible portion during application of the strip to a vaginal surface, and upon release, transferring the tension released on the flexible portion to the arrays of micro-needles, thereby adhering the strip to the vaginal surface.

16. The system of claim 8, wherein a first electrode of the plurality of electrodes is adapted to send an electrical signal to the cervical surface and a second electrode of the plurality of electrodes is adapted to sense an electrical characteristic of the cervical surface based on the transmitted electrical signal.

17. The system of claim 8, further comprising a signal processing unit wirelessly coupled to the continuously wearable device for enhancing signal quality of the electrical impedance and forwarding the enhanced electrical impedance to the analysis system.

18. The system of claim 1, wherein the analysis system is local to the patient.

19. The system of claim 1, wherein the analysis system is geographically separated from the patient.

20. The system of claim 1, wherein the pregnancy-related diagnosis comprises one of labor prediction, prediction of a pregnancy-related complication, and recommendation of a delivery technique.

21. The system of claim 11, wherein the delivery technique includes one of a non-induced vaginal birth, a cesarean section, and a drug-induced labor.

22. The system of claim 11, wherein the pregnancy-related complication include pre-term labor.

23. The system of claim 1, further comprising at least one reservoir for storing a drug therein, wherein the analysis system controls a release of the drug from the reservoir based on an evaluation of the received electrical impedance.

24. The system of claim 23, wherein the analysis system determines at least one of time, duration, and dosage amount of drugs to deliver to the patient by the device.

25. The system of claim 1, wherein the analysis system makes the pregnancy-related diagnosis by comparing the impedance data to data in a database that includes one of historical cervical tissue measurements of the patient and cervical tissue measurements of a plurality of women having similar physiological profiles as the patient.

26. The system of claim 1, wherein the analysis system performs one of delivering medical care to the patient, alerting a medical care provider, and making a medical suggestion to the patient based on an evaluation of the impedance data.

27. A method for characterizing electrical activity of a patient, comprising:
measuring an electrical impedance of a cervical surface of a patient based on a signal applied to the cervical surface by a wearable device adapted to inter-fit within a vaginal region of the patient for a period of time; and
transmitting the measured electrical impedance of the cervical surface to an analysis system for making a pregnancy-related diagnosis.

28. A method of claim 27, wherein the period of time exceeds one month.

29. A method of claim 27, wherein the pregnancy-related diagnosis comprises one of labor prediction, prediction of a pregnancy-related complication, and recommendation of a delivery technique.

30. A method of claim 27, comprising releasing, by the wearable device, drugs for delaying the onset of labor.

31. A method of claim 27, comprising comparing, by the analysis system, received impedance values to a predetermined impedance threshold for performing a Cesarean-section.

32. A method of claim 31, wherein in response to the impedance value being greater than the Cesarean-section threshold, recommending, by the analysis system, the patient have a Cesarean-section at a later date.

33. A method of claim 31, wherein in response to the impedance value being lower than the Cesarean-section threshold, comparing the received impedance value to a threshold for inducing labor in the patient.

34. A method of claim 33, wherein in response to the impedance value being greater than the threshold for inducing labor, recommending, by the analysis system, that labor should be induced in the patient at a later date and wherein in response to the impedance value being less than or equal to the induction threshold, recommending, by the analysis system, inducing labor in the patient.

35. A wearable device comprising:
a reservoir for storing a drug therein;
an electrode for measuring an electrical impedance of a cervical tissue of the patient; and
an analysis system coupled to the wearable device;
wherein the analysis system controls a release of the drug from the reservoir based on the electrical impedance data.

36. The device of claim 35, wherein the reservoir is affixed onto a surface of the wearable device.

37. The device of claim 35, further comprising a micro-needle having one end coupled to the reservoir and another end configured to penetrate into the cervical tissue.

38. A use of a system, comprising:
measuring an electrical impedance of a cervical surface based on a signal applied to the cervical surface by a wearable device adapted to inter-fit within a vaginal region for a period of time; and
transmitting the measured electrical impedance of the cervical surface to an analysis system for making a pregnancy-related diagnosis.

39. The use of claim 38, comprising diagnosing one of labor prediction, prediction of a pregnancy-related complication, and recommendation of a delivery technique.

40. The use of claim 38, comprising releasing drugs by the wearable device for delaying the onset of labor.
